(19) United States
(12) Patent Application Publication
(10) Pub. No.: US 2020/0033584 A1
(43) Pub. Date: Jan. 30, 2020

(54) PATTERN PROJECTOR USING ROTATIONAL SUPERPOSITION OF MULTIPLE OPTICAL DIFFRACTION ELEMENTS AND 3D ENDOSCOPE HAVING THE SAME

(71) Applicant: KOREA ADVANCED INSTITUTE OF SCIENCE AND TECHNOLOGY, Daejeon (KR)

(72) Inventors: Ki-Hun JEONG, Daejeon (KR); Sung-Pyo YANG, Daejeon (KR); Jaebeom KIM, Daejeon (KR)

(21) Appl. No.: 16/521,378

(22) Filed: Jul. 24, 2019

(30) Foreign Application Priority Data
Jul. 24, 2018 (KR) 10-2018-0085917
Jun. 25, 2019 (KR) 10-2019-0075831

(51) Int. Cl. G02B 23/24; A61B 1/00

(52) U.S. Cl. CPC G02B 23/2415; G01B 11/25; A61B 1/00193; A61B 1/00009

(57) ABSTRACT

A subminiature pattern projector using rotational superposition of multiple optical diffraction elements is disclosed. A three-dimensional (3D) endoscope having the pattern projector is also disclosed. The 3D endoscope has a pattern projector that forms a pattern having high density and uniformity for acquiring a 3D image by using an angle offset between two or more optical diffraction elements. The pattern projector irradiates an optical diffraction pattern for shooting the 3D image, or includes a function as illumination for illuminating a region of interest in a human body.

### CROSS-REFERENCE TO RELATED APPLICATIONS

This application claims priority under 35 U.S.C. § 119 to Korean Patent Applications No. 10-2018-0085917, filed on Jul. 24, 2018, and Korean Patent Applications No. 10-2019-0075831, filed on Jun. 25, 2019 in the Korean Intellectual Property Office, the disclosure of which is incorporated herein by reference in its entirety.

### TECHNICAL FIELD

The following disclosure relates to a subminiature pattern projector and a three-dimensional (3D) endoscope having the same.

### BACKGROUND

Typically, an endoscope includes a single tube called a direct endoscope. The endoscope may also be inserted into a patient's body (e.g., inserted through mouth, or through an incision site in surgery) to observe the inside of a human body for treatment and diagnosis so that the doctor may directly see patient's organs with the naked eye during treatment.

This type of endoscope in a single tube provides two-dimensional (2D) image resulting in a lack of cubic effect. This has been causing difficulty to have complete access to the patient's treatment site during delicate surgery. As a solution for the endoscope providing such a 2D image, a 3D endoscope is provided. The disclosure of this section is to provide background of the invention. Applicant notes that this section may contain information available before this application. However, by providing this section, Applicant does not admit that any information contained in this section constitutes prior art.

### SUMMARY

Aspects of the invention provide a subminiature pattern projector using rotational superposition of multiple optical diffraction elements and a three-dimensional (3D) endoscope having the same. Aspects of the invention further provide a 3D image providing endoscope having a pattern projector that forms a pattern having high density and uniformity for acquiring a 3D image by using an angle offset between two or more optical diffraction elements, a pattern projector that irradiates an optical diffraction pattern for shooting a 3D image, or a pattern projector including a function as illumination for illuminating a region of interest in a human body.

An embodiment of the present invention is directed to providing a pattern projector using rotational superposition of multiple optical diffraction elements that may use laser beam and irradiate an optical diffraction pattern having high density and uniformity to a region of interest through adjustment of an angle offset between two or more optical diffraction elements to provide quantitative information of a 3D image for an object.

In addition, an embodiment of the present invention is directed to providing a 3D image providing endoscope having a pattern projector that acquires a 3D image by irradiating an optical diffraction pattern having high density and uniformity formed by a pattern projector using rotational superposition of an optical diffraction element to a region of interest in a human body, and also serves as illumination for illuminating a region of interest in a human body because light provided by a light source part of the pattern projector may be selected as single wavelength laser or illumination light.

In one general aspect, a pattern projector using rotational superposition of multiple optical diffraction elements includes: a light source part outputting a laser beam; and an optical diffraction part including two or more optical diffraction elements and adjusting a rotational angle offset between the optical diffraction elements to generate an arbitrary regular optical diffraction pattern formed while the laser beam passes through the optical diffraction elements.

The optical diffraction elements may be microlens arrays.

The optical diffraction elements may be the microlens arrays formed by forming a plurality of cylindrical cylinder-shaped patterns on a substrate; coating a fluoropolymer thin film on upper portions of the cylindrical cylinder-shaped patterns and a surface of the substrate; performing a heat treatment process for the patterns on which the fluoropolymer thin film is coated; and coating a parylene thin film on the upper portions of the patterns on which the fluoropolymer thin film is coated and the surface of the substrate.

The optical diffraction elements may be the microlens arrays in which hemispherical lenses are continuously arranged on a two-dimensional plane, a fluoropolymer thin film is coated between a spherical surface of the lens and a spherical surface of the lens, and a parylene thin film is coated on the fluoropolymer thin film.

The number of the optical diffraction elements may be two, and the rotational angle offset between the optical diffraction elements may be an offset angle at which the number of overlapping points of a double optical diffraction pattern is selected among angles in a range in which the increase and decrease in a graph changes when the number of the overlapping points of the double light diffraction pattern per unit area according to a rotational angle that is output through the optical diffraction elements by rotating any one of the two optical diffraction elements is represented by the graph.

The rotational angle offset between the optical diffraction elements may be the offset angle selected at an angle at which the number of the overlapping points of the double optical diffraction pattern has a local minimum point in the graph.

The pattern projector may further include a glass substrate or a semiconductor wafer between the optical diffraction elements.

In another general aspect, a three-dimensional (3D) endoscope having a pattern projector using rotational superposition of multiple optical diffraction elements includes: in an endoscope including a tubular body inserted into a region of interest in a human body, a pattern projector module including a laser light source, an illumination light source, and an optical fiber bundle, and including a light source part that allows light output from the laser light source and the illumination light source at one end of the pattern projector module to be output to the other end of the pattern projector module through a coupling with the optical fiber bundle extending from one end to the other end, and an optical diffraction part including two or more optical diffraction elements that diffract the light output from the light source part; and a shooting module collecting a reflected light from the region of interest to form an image when the light output from the pattern projector module irradiates the region of interest, and providing image information, wherein an optical diffraction part adjusts a rotational angle offset between the optical diffraction elements to generate an arbitrary regular optical diffraction pattern formed while the laser beam passes through the optical diffraction elements.

The light source part may be configured to output the light through the optical fiber bundle, and a portion of the optical fiber bundle may be coupled to the laser light source and the other portion of the optical fiber bundle may be coupled to the illustration light source.

The portion of the optical fiber bundle coupled to the laser light source may be located at the center of the optical fiber bundle.

The optical diffraction part may include two or more optical diffraction elements, generate an arbitrary regular optical diffraction pattern through a rotational angle offset between the optical diffraction elements when the laser beam output from the laser light source passes through the optical diffraction elements, and scatter and diffract white light through the rotation angle offset between the optical diffraction elements when the white light output from the illumination light source passes through the optical diffraction elements.

The optical diffraction elements may be microlens arrays.

The optical diffraction elements may be the microlens arrays formed by forming a plurality of cylindrical cylinder-shaped patterns on a substrate; coating a fluoropolymer thin film on upper portions of the cylindrical cylinder-shaped patterns and a surface of the substrate; performing a heat treatment process for the patterns on which the fluoropolymer thin film is coated; and coating a parylene thin film on the upper portions of the patterns on which the fluoropolymer thin film is coated and the surface of the substrate.

The optical diffraction elements may be the microlens arrays in which hemispherical lenses are continuously arranged on a two-dimensional plane, a fluoropolymer thin film is coated between a spherical surface of the lens and a spherical surface of the lens, and a parylene thin film is coated on the fluoropolymer thin film.

The number of the optical diffraction elements may be two, and the rotational angle offset between the optical diffraction elements may be an offset angle at which the number of overlapping points of a double optical diffraction pattern is selected among angles in a range in which the increase and decrease in a graph changes when the number of the overlapping points of the double light diffraction pattern per unit area according to a rotational angle that is output through the optical diffraction elements by rotating any one of the two optical diffraction elements is represented by the graph.

The rotational angle offset between the optical diffraction elements may be the offset angle selected at an angle at which the number of the overlapping points of the double optical diffraction pattern has a local minimum point in the graph.

The laser light source may be a green laser.

The laser light source may be an infrared ray laser.

### BRIEF DESCRIPTION OF THE DRAWINGS

FIG. 1 is an exploded perspective view of a pattern projector using rotational superposition of multiple optical diffraction elements according to an embodiment of the present invention.

FIG. 2 is a conceptual view for describing adjustment of a rotational angle offset between multiple optical diffraction elements according to an embodiment of the present invention.

FIG. 3 is a conceptual view of a process of manufacturing a microlens array, which is an optical diffraction element, according to an embodiment of the present invention.

FIG. 4 is a flowchart of a process of manufacturing a microlens array, which is an optical diffraction element, according to an embodiment of the present invention.

FIG. 5 is a partial enlarged view of a microlens array before parylene coating.

FIG. 6 is a partial enlarged view of a microlens array after parylene coating.

FIG. 7 is a view illustrating an influence of parylene coating on the distribution and uniformity of a pattern.

FIG. 8A is a view illustrating an optical diffraction pattern in a case in which one optical diffraction element is used.

FIG. 8B is a view illustrating an optical diffraction pattern when there is no angle offset in a case in which two optical diffraction elements are used.

FIG. 9 is a graph illustrating the number of superposed pixels of an optical diffraction pattern according to a change in offset angle.

FIG. 10 is a graph illustrating a contrast value of an optical diffraction pattern according to a change in offset angle.

FIG. 11 is a view illustrating optical diffraction patterns at offset angles 0°, 5°, 15°, 22.5°, 30°, and 35° between a plurality of optical diffraction elements.

FIG. 12 is a partial perspective view of a 3D image providing endoscope having a pattern projector according to an embodiment of the present invention.

FIG. 13 is a schematic cross-sectional view of a 3D image providing endoscope having a pattern projector according to an embodiment of the present invention.

FIG. 14 is a schematic cross-sectional view of a pattern projector for an effect according to a switching between a laser light source and an illumination light source according to the present invention.

FIG. 15 is a view illustrating an effect in a case in which white light output from the illumination light source passes through an optical diffraction part in a 3D endoscope according to an embodiment of the present invention.

### DETAILED DESCRIPTION OF EMBODIMENTS

Hereinafter, embodiments of the present invention having a configuration as described above will be described in detail with reference to the accompanying drawings.

A 3D endoscope includes a pair of lenses to provide a stereoscopic image. The 3D endoscope has advantages of providing stereoscopic images of the treatment site to facilitate observation, and facilitating various movements during surgery, to increase the accuracy of mechanical surgery and shorten the operation time.

In one implementation, a 3D endoscopes use a stereoscopic image implementation technology that enables a 3D stereoscopic effect to be perceived by presenting a pair of 2D images having a parallax in both eyes to both eyes of a doctor using the visual difference of both eyes in a stereoscopic method. Therefore, although such a 3D endoscope is easy to implement and is easy to commercialize because it is cheap, it may require an auxiliary mechanism such as stereoscopic glasses and does not provide quantitative information of the 3D image of the patient's treatment site.

In addition, in one implementation of 3D endoscopes a pattern projector module includes a laser diode, a lens part, and a microlens array, and is configured to project pattern light onto an object to acquire 3D data. Since such a pattern projector module has a structure in which the respective necessary components are individually arranged, the pattern projector module may have a large number of components and a complicated process of assembling these components, and the pattern is not clear, which makes it difficult to recognize a clear pattern of a camera, thereby lowering the resolution of acquired data.

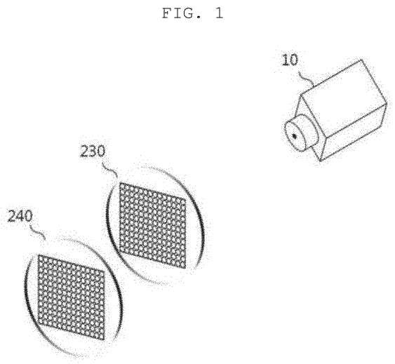

FIG. 1 is an exploded perspective view of a pattern projector using rotational superposition of multiple optical diffraction elements according to an embodiment of the present invention.

Referring to FIG. 1, a pattern projector using rotational superposition of multiple optical diffraction elements according to an embodiment of the present invention includes a light source part 10 and an optical diffraction part 220. The light source part 10 outputs a laser beam having an arbitrary wavelength and is made of a laser having a wavelength selected in a visible light and infrared wavelength range. In addition, the optical diffraction part 220 is disposed to be spaced apart from the light source part 10, and diffracts the laser beam output from the light source part 10 to form an optical diffraction pattern. For example, the optical diffraction part 220 includes two or more optical diffraction elements, and adjusts a rotational angle offset between the optical diffraction elements to generate an arbitrary regular optical diffraction pattern formed while the laser beam output from the light source part passes through the optical diffraction elements.

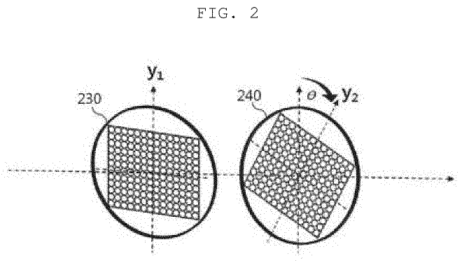

FIG. 2 is a conceptual view for describing adjustment of a rotational angle offset between multiple optical diffraction elements according to an embodiment of the present invention.

Referring to FIG. 2, the optical diffraction part 220 includes two or more optical diffraction elements, and each optical diffraction element has a constant material and structure. As illustrated in FIG. 2, when the respective optical diffraction elements are on a plane formed by two axes perpendicular to each other, a first optical diffraction element 230 through which the beam output from the light source part first passes and a second optical diffraction element 240 spaced apart from the first optical diffraction element by a predetermined interval form an offset angle at which a vertical axis y₁ and an axis y₂ of each element are shifted by θ. Therefore, the laser beam output from the light source part rotates at a predetermined offset angle and passes through the superposed optical diffraction elements, thereby forming a diffraction pattern. The respective optical diffraction elements are spaced apart from each other by a predetermined interval, and may have a glass substrate (e.g., cover glass) or a semiconductor wafer interposed therebetween.

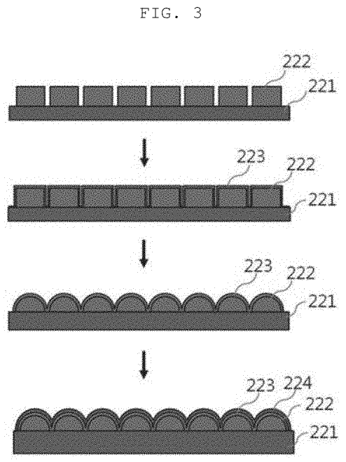

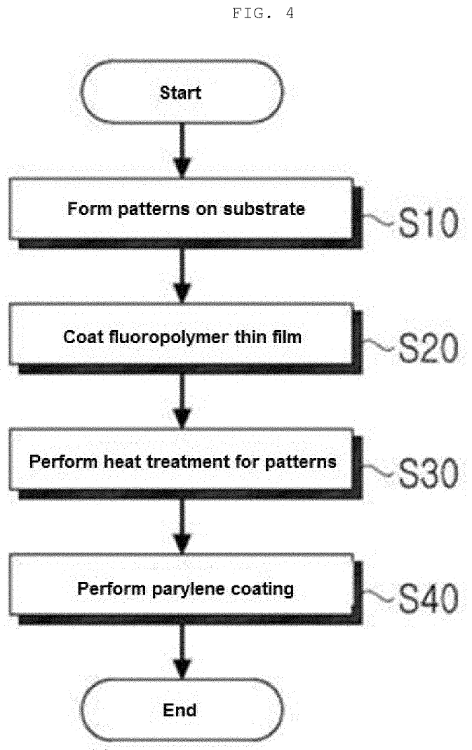

FIG. 3 is a conceptual view of a process of manufacturing a microlens array, which is an optical diffraction element, according to an embodiment of the present invention and FIG. 4 is a flowchart of a process of manufacturing a microlens array, which is an optical diffraction element, according to an embodiment of the present invention.

Referring to FIGS. 3 and 4, the optical diffraction elements 230 and 240 included in the optical diffraction part 220 may be microlens arrays. The microlens array, which is the optical diffraction element, may be manufactured by a process described below. First, cylindrical cylinder-shaped patterns 222 are formed on a substrate 221 (S10). In this case, the substrate 221 may be formed of a glass substrate, a silicon wafer, or the like. In addition, the cylindrical cylinder-shaped patterns 222 are photo resist or polymer patterns, and may be generally formed through a semiconductor etching or photolithography process. Thereafter, a fluoropolymer thin film 223 is coated on upper surfaces of the patterns 222 and a surface of the substrate 221 (S20). The fluoropolymer thin film 223 may be formed of various precursors such as C₃F₈, C₄F₈, CHF₃, and the like. Next, if a heat treatment process is performed for the patterns on which the fluoropolymer thin film is coated as a thermal reflow process (S30), e.g., if the patterns are heated at a temperature which is a glass transition temperature Tg or more of the photoresist or polymer, a spherical shaped lens is formed while a process of reducing a surface area of the photoresist or polymer occurs. A parylene coating is performed for a surface of the microlens array having the spherical shape formed by the heat treatment (S40).

In the microlens array according to an embodiment of the present invention, continuous hemispherical lenses may be arranged in a rectangular or hexagonal array in a matrix structure on a two-dimensional plane, the fluoropolymer thin film may be coated between a spherical surface of the lens and a spherical surface of the lens, and a parylene thin film may be coated on the fluoropolymer thin film.

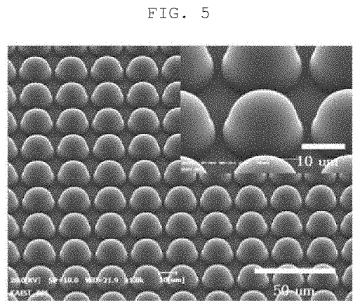

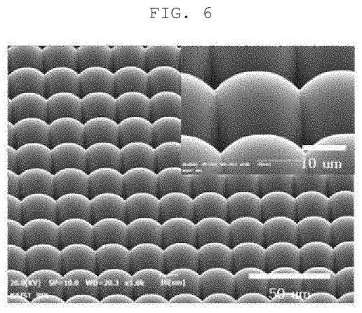

FIG. 5 illustrates a partial enlarged view of a microlens array before parylene coating and FIG. 6 is a partial enlarged view of a microlens array after parylene coating.

When the laser beam passes through the microlens array, the laser beam is diffracted to form diffracted pattern light. As a filling rate of the microlens array is larger, the diffraction pattern shows a uniform intensity distribution. In embodiments, there are characteristics that the diffraction is well performed, the intensity of the diffracted light is not concentrated on the diffraction order of 0, and a change in intensity according to an increase in the diffraction order is small. The diffraction order of 0 means that straight light proceeds as it is through the diffraction element. When an energy ratio of the light proceeding as the straight light among the total energy of an incident light is increased, the uniformity of the overall pattern is lowered, and when the energy ratio of the light proceeding as the straight light is decreased, the uniformity of the overall pattern is increased.

In addition, as a curvature of the microlens array is larger, refraction of light occurs well. As a result, more light energy is transferred to a pattern of high diffraction order, thereby making it possible to form a pattern having a wide range of energy distribution.

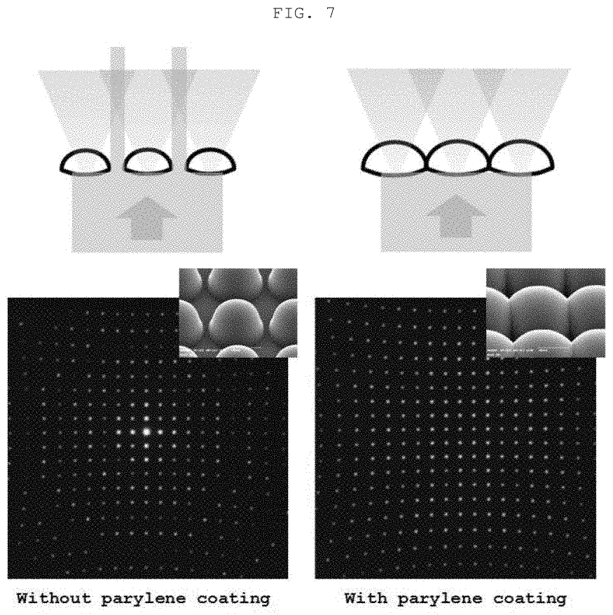

FIG. 7 is a view illustrating an influence of the parylene coating of the microlens array on the distribution and uniformity of a pattern.

Referring to FIG. 7, it may be seen that when the parylene coating is not performed, the energy of the laser beam passes through the microlens array by the straight light without refraction, and a portion where the energy is concentrated on the diffraction order of 0 is generated. On the contrary, it may be seen that when the parylene coating is performed, the filling rate of the microlens array is increased and most of the light is refracted at the surface of the microlens array, which results in reducing the portion where the energy is concentrated on the diffraction order of 0, and the uniformity of the pattern is increased as compared with the case in which the parylene coating is not performed.

In embodiments, the microlens array, which is the optical diffraction element, according to an embodiment of the present invention may further increase the filling rate of the microlens array through the parylene coating, and when the microlens array for which the parylene coating is performed is used as the optical diffraction element, the optical diffraction pattern for acquiring the 3D image has high density and uniformity.

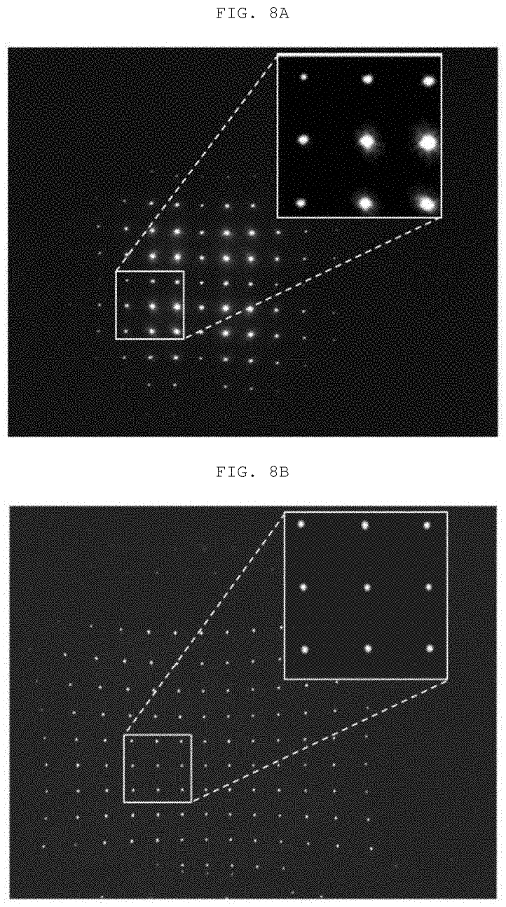

FIGS. 8A and 8B are views illustrating that a pattern projector has an optical diffraction pattern of high uniformity when multiple optical diffraction elements are used. FIG. 8A is a view illustrating an optical diffraction pattern in a case in which one optical diffraction element is used and FIG. 8B is a view illustrating an optical diffraction pattern when there is no angle offset (i.e., an offset angle is 0°) in a case in which two optical diffraction elements are used.

Referring to FIG. 8B, it may be seen that a field of view (FOV) is increased as compared with FIG. 8A.

In addition, referring to enlarged portions of FIGS. 8A and 8B, it may be seen that, when the two optical diffraction elements are used compared to when one optical diffraction element is used, the uniformity of the optical diffraction pattern is increased as the refraction occurs twice on the surface of the microlens array. For example, since it is possible to form a pattern having a wide field of view and high uniformity with a laser point light source according to embodiments of the present invention by using only the two or more optical diffraction elements, a separate device for diffusing the light source to form the pattern having the wide field of view and high uniformity is unnecessary. As a result, a structure of the pattern projector may be simplified and miniaturized, and cost may be reduced.

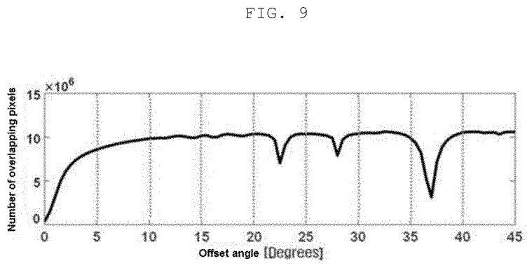

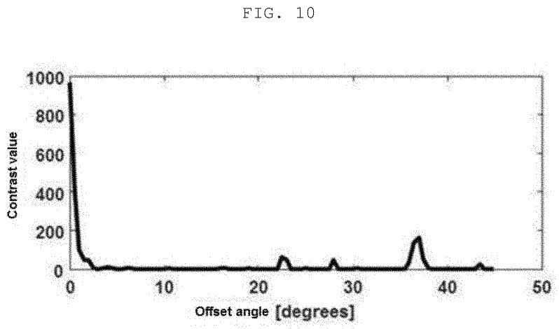

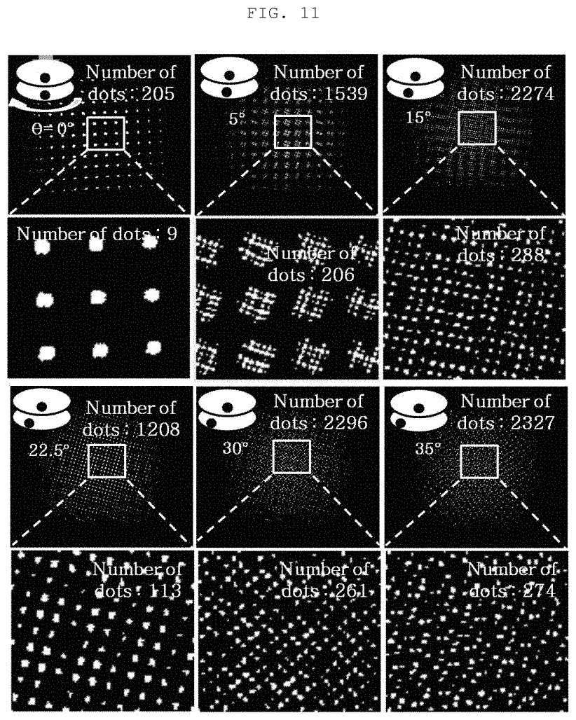

FIG. 9 is a graph illustrating the number of superposed pixels of an optical diffraction pattern according to a change in offset angle, FIG. 10 is a graph illustrating a contrast value of an optical diffraction pattern according to a change in offset angle, and FIG. 11 is a view illustrating optical diffraction patterns at offset angles 0°, 5°, 15°, 22.5°, 30°, and 35° between a plurality of optical diffraction elements.

FIGS. 9 to 11 relate to the optical diffraction patterns according to a change in the offset angle between the two optical diffraction elements formed by rotating the second optical diffraction element in the range of 0° to 45° as illustrated in FIG. 2 using the two optical diffraction elements. Referring to FIG. 9, in a case in which the offset angles between the optical diffraction elements are 22.5°, 28°, and 37°, it may be seen that the number of superposed pixels of the optical diffraction pattern is significantly reduced. In addition, referring to FIG. 10, in a case in which the offset angles between the optical diffraction elements are 22.5°, 28°, and 37°, it may be seen that the contrast value of the optical diffraction pattern has the maximum value. In addition, referring to FIG. 11, in a case in which the offset angle between the optical diffraction elements is 22.5°, it may be seen that the number of dots of the optical diffraction pattern is significantly reduced, but the contrast of the pattern is clearly distinct. Since the pattern projector module according to an embodiment of the present invention is intended to increase the resolution for a 3D image of an object and acquire a quantitative profile for the 3D image by irradiating the optical diffraction pattern output from the pattern projector onto the object, the optical diffraction pattern needs to have high density and uniformity simultaneously. In embodiments, the number of dots of the pattern may need to be large in the unit area, and the intensity of the optical diffraction pattern of a high order may need to be strong such that the contrast is good. In FIGS. 9 and 10, the optical diffraction patterns formed at the offset angles 22.5°, 28°, and 37° between the optical diffraction elements have high density and uniformity. The offset angle 22.5° or 28° between the optical diffraction elements shows characteristics that since the number of patterns per unit area is large and there are few overlapping points forming the pattern, a clear pattern is formed. The offset angle 37° shows characteristics that since the overlapping of the points forming the pattern is significantly reduced, a very clear pattern is formed, but has density somewhat lower than that of the offset angle 22.5° or 28°. However, considering a distance between the object to which the optical diffraction pattern is irradiated by the pattern projector and the optical diffraction part, when the distance is short, the offset angle 37° may be advantageous compared to the offset angles 22.5° and 28°.

In the pattern projector using the rotational superposition of the multiple optical diffraction elements according to an embodiment of the present invention, the rotational angle offset between the optical diffraction elements may vary depending on a wavelength of the laser output from the light source part, and when the number of overlapping points of a double light diffraction pattern per unit area according to the rotational angle that is output through the optical diffraction elements by rotating any one of the two optical diffraction elements is represented by a graph, the number of overlapping points of the double optical diffraction pattern may be selected among angles in a range in which the increase and decrease in the graph changes. Furthermore, the rotational angle offset between the optical diffraction elements is preferably an offset angle selected from an angle having a local minimum point in the graph in view of high density and uniformity of the optical diffraction pattern. Here, the minimum value means an offset angle at which the increase and decrease in the number of points of the optical diffraction pattern per unit area changes with an increase in the offset angle when the number of points of the optical diffraction pattern per unit area that is output through the optical diffraction elements according to the offset angle between the optical diffraction elements is represented by a graph. In addition, the number of points of the optical diffraction pattern per unit area that is output through the optical diffraction elements with the change in the offset angle may be represented by the graph, but the number of overlapping pixels of the optical diffraction pattern that is output through the optical diffraction element with the change in the offset angle may be represented by the graph to determine an optimal offset angle.

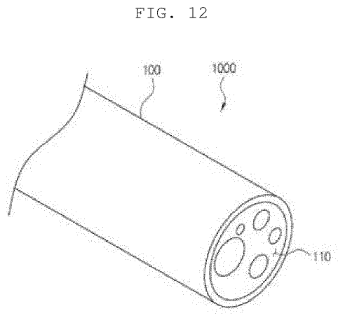

FIG. 12 illustrates a partial perspective view of a 3D image providing endoscope having a pattern projector according to an embodiment of the present invention.

Referring to FIG. 12, a 3D image providing endoscope 1000 having a pattern projector is configured to include a tubular body 100, an operation part formed at one end of the body, and a search part 110 formed at the other end of the body and inserted into the human body to perform observation, examination, and surgery. The body 100 may have a typical endoscope configuration in which an operation part is formed at one end thereof and a search part 110 is formed at the other end thereof, and FIG. 12 illustrates the other end of the endoscope.

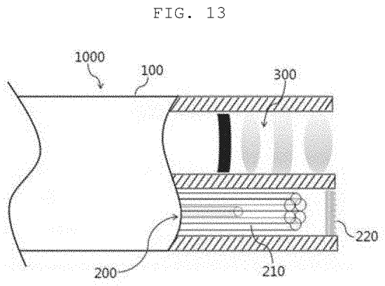

FIG. 13 illustrates a schematic cross-sectional view of a 3D image providing endoscope having a pattern projector according to an embodiment of the present invention.

Referring to FIG. 13, the endoscope 1000 according to embodiments of the present invention includes a pattern projector module 200 and a shooting module 300. The pattern projector module 200 includes a laser light source, an illumination light source, and an optical fiber bundle, and has a light source part 210 that allows the light output from the laser light source and the illumination light source at one end of the pattern projector module 200 to be output to the other end of the pattern projector module 200 through the optical fiber bundle extending from one end thereof to the other end thereof, and an optical diffraction part 220 that diffracts the light output from the light source part. The shooting module 300 collects the reflected light from the region of interest to form an image when the light output from the pattern projector module irradiates the region of interest in the human body, and then provides image information to a display device connected to one end of the endoscope.

In the pattern projector module 200, the light output from the light source part 210 may include the laser and illumination light, and a switch for turning on and off the laser light source and the illumination light source is formed at one end of the endoscope according to embodiments of the present invention. The light output from the laser light source and the illumination light source is emitted through the optical fiber bundle. A portion or at least one optical fiber bundle is coupled to a single wavelength of laser light and the other portion of the optical fiber bundle is coupled to the illumination light. Here, one or a portion of the optical fiber bundle coupled to the laser light is preferably an optical fiber located at the center of the optical fiber bundle.

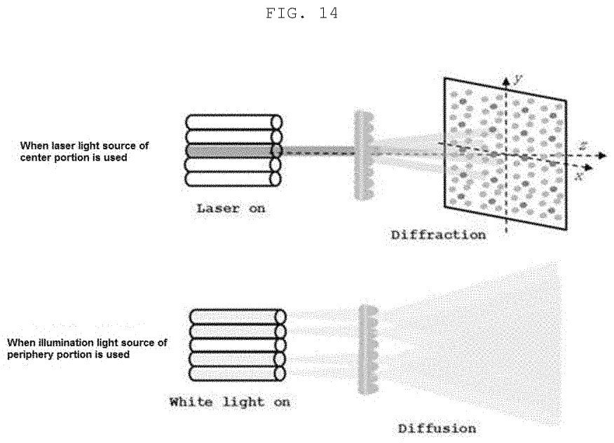

FIG. 14 illustrates a schematic cross-sectional view of a pattern projector for an effect according to a switching between a laser light source and an illumination light source according to embodiments of the present invention.

Referring to FIG. 14, when the switch of the laser light source in the light source part 210 is turned on, the laser beam coupled to the optical fiber is output, and the laser beam passing through the optical fiber has a characteristic that an output position is fixed and it is a circular parallel beam. The output laser light passes through the optical diffraction part 220 to become pattern light having an arbitrary regular light diffraction pattern, and is irradiated to the region of interest in the human body. When the irradiated pattern light is reflected on the region of interest, the shooting module collects the reflected light to form an image, and provides 3D image information of the region of interest to a display device connected to one end of the endoscope. A user quantitatively acquires information on a profile of the 3D image of the region of interest in the human body. Here, since the region of interest in the human body to obtain the 3D image information is mainly red, in case of the pattern light by a red laser, it is difficult to visually confirm whether the pattern light is well irradiated to the region of interest. Therefore, the laser beam of a single wavelength is preferably a green light whose wavelength region is selected between 500 nm and 570 nm. In addition, the laser beam of the single wavelength may be an infrared ray having a wavelength longer than 700 nm. When the region of interest in the human body is sensitive to visible light, an infrared laser may be used.

In addition, when the switch of the illumination light source in the light source part 210 is turned on, the illumination light coupled to the optical fiber is output through the optical fiber, and the output illumination light passes through the optical diffraction part to illuminate the region of interest in the human body.

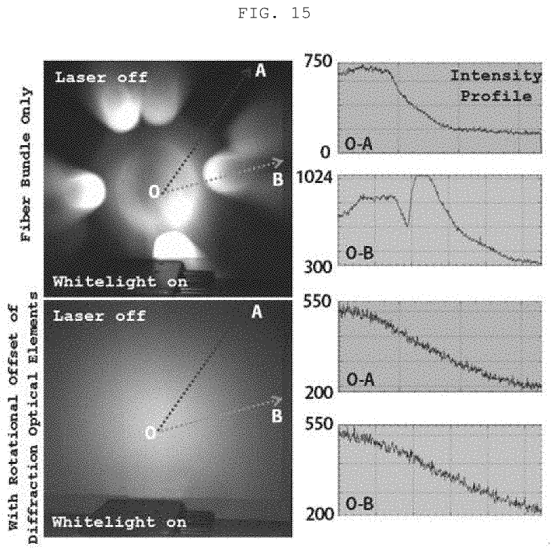

FIG. 15 is a view illustrating an effect in a case in which white light output from the illumination light source passes through a light diffraction part in a 3D endoscope according to an embodiment of the present invention.

Referring to FIG. 15, it may be seen that a uniform illumination having a wide angle of view is formed as compared with a case in which the white light passes through only the optical fiber bundle because the white light passes through the optical diffraction part 220 to which the offset is applied and the optical diffraction part 220 serves as a diffuser, when the white light is output as illumination.

The optical diffraction part 220 of the pattern projector module 200 of the 3D endoscope includes two or more optical diffraction elements, and adjusts a rotational angle offset between the optical diffraction elements to generate an arbitrary regular optical diffraction pattern formed while the laser beam output from the light source part passes through the optical diffraction elements. In embodiments, the optical diffraction part 220 of the pattern projector module 200 of the 3D endoscope has the same configuration and characteristics as those of the optical diffraction part 220 of the pattern projector using the rotational superposition of the multiple optical diffraction elements. Therefore, the optical diffraction part 220 of the pattern projector module 200 of the 3D endoscope may be replaced with a description of the optical diffraction part 220 of the pattern projector using the rotational superposition of the multiple optical diffraction elements.

Since the pattern projector module 200 is manufactured within a diameter of 2.7 mm, a sectional area of the 3D image providing endoscope having the pattern projector may be reduced. As the diameter of the endoscope is minimized, the endoscope may be easily inserted into the human body, and an incision site is minimized when the endoscope is inserted by incising the human body, resulting in a rapid recovery rate of the patient after the operation.

Although embodiments of the present invention have been described, it will be understood by those skilled in the art that various modifications and equivalent other embodiments are possible from embodiments of the present invention.

For example, the pattern projector according to embodiments of the present invention may be used for 3D shape restoration or depth measurement in the field of 3D imaging systems as well as the 3D endoscope according to embodiments of the present invention.

The pattern projector according to embodiments of the present invention may irradiate the optical diffraction pattern having high density and uniformity through the rotational superposition that adjusts the angle offset between the two or more optical diffraction elements.

In addition, the optical diffraction pattern having high density and uniformity is irradiated, thereby making it possible to increase the resolution of the 3D image data of the region of interest and acquire the quantitative information on the 3D image data.

The 3D image providing endoscope having the pattern projector according to embodiments of the present invention may irradiate the optical diffraction pattern having high density and uniformity through the rotational superposition that adjusts the angle offset between the two or more optical diffraction elements, thereby increasing the resolution of the 3D image data of the region of interest in the human body and acquiring the quantitative information on the 3D image data.

In addition, since the light source of the pattern projector includes the single wavelength laser and the illumination light, the pattern projector may acquire the 3D image data when the single wavelength laser is selected, and may serve as the illumination for illuminating the region of interest in the human body when the white light, which is the illumination light, is selected. In this case, the optical diffraction element may form uniform illumination having a wide optic angle by scattering and diffracting the white light when the white light passes therethrough.

In addition, as a diameter of the endoscope is minimized by using the pattern projector having two functions as the light source of the endoscope and acquiring the 3D image through a single lens, the endoscope may be easily inserted into the human body, and the incision site is minimized when the endoscope is inserted by incising the human body, resulting in a rapid recovery rate of the patient after the operation.

Accordingly, the actual technical protection scope of the present invention should be defined by the technical idea of the following claims.

### DETAILED DESCRIPTION OF MAIN ELEMENTS

| | |
|---|---|
| 1000: endoscope | |
| 100: body | |
| 110: search part | |
| 200: pattern projector module | |
| 210: light source part | 220: optical diffraction part |
| 221: substrate | 222: pattern |
| 223: fluoropolymer thin film | 224: parylene thin film |
| 230: first optical diffraction element | 240: second optical diffraction |
| | element |
| 300: shooting module | |

What is claimed is:

1. A pattern projector using rotational superposition of multiple optical diffraction elements, the pattern projector comprising:
a light source part outputting a laser beam; and
an optical diffraction part including two or more optical diffraction elements and adjusting a rotational angle offset between the optical diffraction elements to generate an arbitrary regular optical diffraction pattern formed while the laser beam passes through the optical diffraction elements.

2. The pattern projector of claim 1, wherein the optical diffraction elements are microlens arrays.

3. The pattern projector of claim 2, wherein the optical diffraction elements are the microlens arrays formed by
forming a plurality of cylindrical cylinder-shaped patterns on a substrate;
coating a fluoropolymer thin film on upper portions of the cylindrical cylinder-shaped patterns and a surface of the substrate;
performing a heat treatment process for the patterns on which the fluoropolymer thin film is coated; and
coating a parylene thin film on the upper portions of the patterns on which the fluoropolymer thin film is coated and the surface of the substrate.

4. The pattern projector of claim 2, wherein the optical diffraction elements are the microlens arrays in which hemispherical lenses are continuously arranged on a two-dimensional plane, a fluoropolymer thin film is coated between a spherical surface of the lens and a spherical surface of the lens, and a parylene thin film is coated on the fluoropolymer thin film.

5. The pattern projector of claim 1, wherein the number of the optical diffraction elements is two, and the rotational angle offset between the optical diffraction elements is an offset angle at which the number of overlapping points of a double optical diffraction pattern is selected among angles in a range in which the increase and decrease in a graph changes when the number of the overlapping points of the double light diffraction pattern per unit area according to a rotational angle that is output through the optical diffraction elements by rotating any one of the two optical diffraction elements is represented by the graph.

6. The pattern projector of claim 5, wherein the rotational angle offset between the optical diffraction elements is the offset angle selected at an angle at which the number of the overlapping points of the double optical diffraction pattern has a local minimum point in the graph.

7. The pattern projector of claim 1, further comprising a glass substrate or a semiconductor wafer between the optical diffraction elements.

8. A three-dimensional (3D) endoscope having a pattern projector, the 3D endoscope comprising: in an endoscope including a tubular body inserted into a region of interest in a human body,
a pattern projector module including a laser light source, an illumination light source, and an optical fiber bundle, and including a light source part that allows light output from the laser light source and the illumination light source at one end of the pattern projector module to be output to the other end of the pattern projector module through a coupling with the optical fiber bundle extending from one end to the other end, and an optical diffraction part including two or more optical diffraction elements that diffract the light output from the light source part; and
a shooting module collecting a reflected light from the region of interest to form an image when the light output from the pattern projector module irradiates the region of interest, and providing image information,
wherein an optical diffraction part adjusts a rotational angle offset between the optical diffraction elements to generate an arbitrary regular optical diffraction pattern formed while the laser beam passes through the optical diffraction elements.

9. The 3D endoscope of claim 8, wherein the light source part is configured to output the light through the optical fiber bundle, and
a portion of the optical fiber bundle is coupled to the laser light source and the other portion of the optical fiber bundle is coupled to the illustration light source.

10. The 3D endoscope of claim 9, wherein the portion of the optical fiber bundle coupled to the laser light source is located at the center of the optical fiber bundle.

11. The 3D endoscope of claim 10, wherein the optical diffraction part includes two or more optical diffraction elements, generates an arbitrary regular optical diffraction pattern through a rotational angle offset between the optical diffraction elements when the laser beam output from the laser light source passes through the optical diffraction elements, and scatters and diffracts white light through the rotation angle offset between the optical diffraction elements when the white light output from the illumination light source passes through the optical diffraction elements.

12. The 3D endoscope of claim 11, wherein the optical diffraction elements are microlens arrays.

13. The 3D endoscope of claim 12, wherein the optical diffraction elements are the microlens arrays formed by
forming a plurality of cylindrical cylinder-shaped patterns on a substrate;
coating a fluoropolymer thin film on upper portions of the cylindrical cylinder-shaped patterns and a surface of the substrate;
performing a heat treatment process for the patterns on which the fluoropolymer thin film is coated; and
coating a parylene thin film on the upper portions of the patterns on which the fluoropolymer thin film is coated and the surface of the substrate.

14. The 3D endoscope of claim 12, wherein the optical diffraction elements are the microlens arrays in which hemispherical lenses are continuously arranged on a two-dimensional plane, a fluoropolymer thin film is coated between a spherical surface of the lens and a spherical surface of the lens, and a parylene thin film is coated on the fluoropolymer thin film.

15. The 3D endoscope of claim 11, wherein the number of the optical diffraction elements is two, and the rotational angle offset between the optical diffraction elements is an offset angle at which the number of overlapping points of a double optical diffraction pattern is selected among angles in a range in which the increase and decrease in a graph changes when the number of the overlapping points of the double light diffraction pattern per unit area according to a rotational angle that is output through the optical diffraction elements by rotating any one of the two optical diffraction elements is represented by the graph.

16. The 3D endoscope of claim 15, wherein the rotational angle offset between the optical diffraction elements is the offset angle selected at an angle at which the number of the overlapping points of the double optical diffraction pattern has a local minimum point in the graph.

17. The 3D endoscope of claim 8, wherein the laser light source is a green laser.

18. The 3D endoscope of claim 8, wherein the laser light source is an infrared ray laser.
